(19) United States
(12) Patent Application Publication
(10) Pub. No.: US 2025/0327790 A1
(43) Pub. Date: Oct. 23, 2025

(54) RESIDUAL STRESS MEASUREMENT METHOD AND SYSTEM FOR CERAMIC MATERIALS

(71) Applicants: DONGGUAN CITY WONDERFUL CERAMICS INDUSTRIAL PARK CO., LTD., Dongguan, Guangdong (CN); CENTRE OF EXCELLENCE FOR ADVANCED MATERIALS, Dongguan, Guangdong (CN); MARCOPOLO HOLDINGS CO., LTD., Dongguan, Guangdong (CN)

(72) Inventors: Hailong YU, Dongguan (CN); Jiaqi HUANG, Dongguan (CN); Delin RAO, Dongguan (CN); Kehui LIN, Dongguan (CN); Xiaodong HOU, Dongguan (CN); Ruiyao ZHANG, Dongguan (CN); Shuyan ZHANG, Dongguan (CN); Zhongmin LI, Dongguan (CN)

(73) Assignees: DONGGUAN CITY WONDERFUL CERAMICS INDUSTRIAL PARK CO., LTD., Dongguan, Guangdong (CN); CENTRE OF EXCELLENCE FOR ADVANCED MATERIALS, Dongguan, Guangdong (CN); MARCOPOLO HOLDINGS CO., LTD., Dongguan, Guangdong (CN)

(21) Appl. No.: 18/562,377

(22) PCT Filed: Feb. 16, 2023
(86) PCT No.: PCT/CN2023/076520

§ 371 Date: Dec. 18, 2024

(30) Foreign Application Priority Data
Aug. 16, 2022 (CN) 202210979980.9

(51) Int. Cl. G01N 33/38; B23K 26/0622; B23K 26/352; B23K 103/00; G01N 3/02

(52) U.S. Cl. CPC G01N 33/388; B23K 26/0622; B23K 26/3576; G01N 3/02; B23K 2103/52

(57) ABSTRACT

The present disclosure provides a residual stress measurement method and system for ceramic materials. The residual stress measurement method for ceramic materials comprises the following steps: polishing a target position by using a laser engraving machine; after polishing is completed, pasting a strain rosette at the target position, and connecting the pasted strain rosette with a strain collector; designing a target punching path according to a punching position of the strain rosette in a software terminal of the laser engraving machine; punching the ceramic materials to be measured according to the target punching path by using the laser engraving machine; after punching is completed, inputting collected strain data into a residual stress analysis terminal by a strain collector, and calculating and obtaining residual stress result data by the residual stress analysis terminal.

### TECHNICAL FIELD

The present disclosure relates to the technical field of ceramic material performance measurement, in particular to a residual stress measurement method and system for ceramic materials.

### BACKGROUND

Due to uneven mechanical deformation, temperature change and phase change caused by manufacturing process, uneven plastic deformation is usually caused for materials. After external effects are removed, due to residual plastic deformation on the materials, corresponding elastic deformation can be caused in the material to keep a balanced state of components and stress is generated in the materials. The stress is called internal stress. Internal stress exists in a large area of the materials and is considered to be uniform, and related internal force and internal moment keep balance on various sections of an object. The stress is called internal stress residual stress. Through the existence of residual stress, the materials are easy to cause deformation, cracking and other phenomena in the using process, and the service life of the materials is affected.

There are two main kinds of residual stress measurement methods: nondestructive measurement and destructive measurement. These methods are mature in the measurement of metal components and materials, and corresponding standards are also formed. However, there is no relevant standard and mature measurement method for residual stress measurement of ceramic materials at present. Especially, with the rise of building ceramics materials such as rock slabs, due to the existence of residual stress rock slabs, the rock slabs are easy to damage in the processing and using processes, and the development of these materials is seriously restricted.

As an effective residual stress measurement method, the blind hole method is very mature in the application of metal materials. According to the principle, a small hole is punched on the surface of the materials with a drill bit to release the stress at a measured point, and then the released residual stress is calculated and obtained through the deformation induced by strain gages. However, due to high hardness and wear resistance of ceramic materials, ordinary drill bits cannot punch holes. Even if diamond drill bits are used, measurement results are inaccurate due to large calorific value. At the same time, the drill bits are expensive and high in cost, so that the current blind hole method is difficult to be applied to the residual stress measurement of ceramic materials. Therefore, there is no residual stress measurement method suitable for ceramic materials in the prior art.

Therefore, the prior art has defects and needs to be improved and developed.

### SUMMARY

The technical problem to be solved by the present disclosure is that aiming at the defects in the prior art, the present disclosure provides a residual stress measurement method and system for ceramic materials, and the problem that there is no residual stress measurement method suitable for ceramic materials in the prior art is solved.

The technical scheme adopted for solving the technical problem of the present disclosure is as follows.

Disclosed is a residual stress measurement method for ceramic materials. The residual stress measurement method for ceramic materials is realized based on a residual stress measurement system for ceramic materials. The residual stress measurement system for ceramic materials includes a laser engraving machine, a strain collector, and a residual stress analysis terminal connected with the strain collector.

The residual stress measurement method for ceramic materials includes the following steps:
polishing a target position of a strain rosette to be pasted on the surface of ceramic materials to be measured by the laser engraving machine;
after polishing is completed, pasting the strain rosette on the surface of the ceramic materials to be measured corresponding to the target position, and connecting the pasted strain rosette with the strain collector;
designing a target punching path according to a punching position of the strain rosette in a software terminal of the laser engraving machine;
punching the ceramic materials to be measured according to the target punching path by using the laser engraving machine; and
after punching is completed, inputting collected strain data into a residual stress analysis terminal by the strain collector, and calculating and obtaining residual stress result data by the residual stress analysis terminal.

In an implementation method, the step of polishing a target position of a strain rosette to be pasted on the surface of ceramic materials to be measured by the laser engraving machine includes the following steps:
placing the ice box on a workbench of a laser engraving machine, the ice box is filled with ice cubes;
placing the ceramic materials to be measured on the ice box, and setting a position to be punched at a middle position of the ice box; and
turning on the laser engraving machine, and polishing a target position of a strain rosette to be pasted on the surface of the ceramic materials to be measured;
Wherein, during polishing, the laser wavelength is 355 nm, the pulse width is 5-30 ns, the frequency is 5-30 HZ, the power is 3-30 W, and the number of times of polishing is 1-5.

In an implementation method, the step of after polishing is completed, pasting the strain rosette on the surface of the ceramic materials to be measured corresponding to the target position, and connecting the pasted strain rosette with the strain collector includes the following steps:
after polishing is completed, pasting the strain rosette on the surface of the ceramic materials to be measured corresponding to the target position through glue; and
fixing the position of the strain rosette by using annular terminals, and connecting the strain rosette with the strain collector;
wherein the strain rosette includes three strain elements, and the angles of sensitive grids are 0 °, 45° and 90°, respectively.

In an implementation method, before the step of designing a punching path according to a punching position of the strain rosette in a software terminal of the laser engraving machine, the method also includes the following steps:
inputting the elastic modulus and Poisson's ratio of the ceramic materials to be measured in the residual stress analysis terminal on the residual stress analysis terminal.

In an implementation method, the step of designing a target punching path according to a punching position of the strain rosette in a software terminal of the laser engraving machine includes the following steps:
turning on the laser engraving machine, and adjusting the position of a laser head to the punching position aligned with the strain rosette;
designing the target punching path, and carrying out focusing operation;
wherein the laser wavelength of the laser engraving machine is 355 nm, a 3D (three-dimensional) galvanometer scanning head is adopted, the diameter of target punching size is 0.5-3 mm, and the depth of target punching size is 1.2 times of the diameter.

In an implementation method, the step of designing a target punching path specifically includes the following steps:
in the software terminal of the laser engraving machine, according to the target punching size, making a first circular ring path with the same diameter as the target punching size;
selecting internal filling, setting line angles to be 45°, setting line spacings to be 0.01 mm, and selecting evenly distributed filling lines;
selecting the filled first circular ring path to carry out overall calculation, and winding once along the first circular ring path;
making a second circular ring path so that the diameter of the second circular ring path is smaller than the target punching size;
selecting internal filling, setting line angles to be 135° so that lines are mutually perpendicular to laser lines of the first circular ring path, setting line spacings to be 0.01 mm, and selecting evenly distributed filling lines;
selecting the filled second circular ring path to carry out overall calculation, and winding once along the second circular ring path; and
overlapping the centers of the first circular ring path and the second circular ring path to complete the design of the punching path.

In an implementation method, the residual stress measurement system for ceramic materials also includes an air draft device used for eliminating powder generated by the laser engraving machine during punching.

The step of punching the ceramic materials to be measured according to the target punching path by using the laser engraving machine includes the following steps:
punching the ceramic materials to be measured according to the target punching path by using the laser engraving machine, and turning on the air draft device at the same time.

The laser power of the laser engraving machine is 3-30 W, the diameter of focused pulsed beams is 0.02-0.05 mm, the pulse width is 5-30 ns, and the frequency is 5-30 HZ.

The suction of the air draft device is 1-10 KPa, the air volume is 10-100 m³/h, and the distance between a suction opening of the air draft device and the laser drilling position is 0.1-1 m.

The laser engraving machine is in a discontinuous working mode, and continuously works for 1-10 cycles each time according to set parameters. The depth of interaction is 0.005-0.05 mm each time, and the next operation is carried out at an interval of 10-60 seconds after each interaction until the depth of the target punching size is reached.

In an implementation method, the step of after punching is completed, inputting collected strain data into a residual stress analysis terminal by the strain collector, and calculating and obtaining residual stress result data by the residual stress analysis terminal includes the following steps:
after punching is completed, collecting stabilized strain values by the strain collector;
obtaining the strain values by the residual stress analysis terminal; and
calculating and obtaining residual stress result data according to the elastic modulus and Poisson's ratio by the residual stress analysis software in the residual stress analysis terminal.

In an implementation method, the residual stress result data include maximum principal stress, minimum principal stress, an included angle of 0 degree and equivalent stress.

The present disclosure also provides a residual stress measurement system for ceramic materials, including:
a laser engraving machine, used for polishing a target position of a strain rosette to be pasted on the surface of the ceramic materials to be measured, and punching the ceramic materials to be measured according to the target punching path;
a strain collector, used for connecting a strain rosette pasted on the surface of the ceramic materials to be measured corresponding to the target position after polishing is completed, and inputting collected strain data into a residual stress analysis terminal after punching is completed;
a residual stress analysis terminal, connected with the strain collector and used for collecting released strain and calculating and obtaining residual stress result data; and
an air draft device, used for eliminating powder generated during punching of the laser engraving machine.

The present disclosure provides a residual stress measurement method and system for ceramic materials. The residual stress measurement method is realized based on the residual stress measurement system for ceramic materials. The residual stress measurement method for ceramic materials includes a laser engraving machine, a strain collector, and a residual stress analysis terminal connected with the strain collector. The residual stress measurement method for ceramic materials includes the following steps: polishing a target position of a strain rosette to be pasted on the surface of the ceramic materials to be measured by using the laser engraving machine; after polishing is completed, pasting the strain rosette on the surface of the ceramic materials to be measured corresponding to the target position, and connecting the pasted strain rosette with the strain collector; designing a target punching path according to a punching position of the strain rosette in a software terminal of the laser engraving machine in a software terminal of the laser engraving machine: punching the ceramic materials to be measured according to the target punching path by using the laser engraving machine: after punching is completed, inputting collected strain data into the residual stress analysis terminal by the strain collector, and calculating and obtaining residual stress result data by the residual stress analysis terminal. The ceramic materials are punched by the laser engraving machine, and the residual stress result data are automatically calculated by the residual stress analysis terminal, so that the residual stress measurement of the ceramic materials is realized and can be used for the production guidance of the ceramic material, and then the production quality of ceramic products is improved to solve risks and hidden dangers existing in the later using process.

### BRIEF DESCRIPTION OF THE DRAWINGS

FIG. 1 is a flow chart of a preferred embodiment of a residual stress measurement method for ceramic materials in the present disclosure.

FIG. 2 is a specific flow chart of step S100 in a preferred embodiment of a residual stress measurement method for ceramic materials in the present disclosure.

FIG. 3 is a specific flow chart of step S200 in a preferred embodiment of a residual stress measurement method for ceramic materials in the present disclosure.

FIG. 4 is a specific flow chart of step S300 in a preferred embodiment of a residual stress measurement method for ceramic materials in the present disclosure.

FIG. 5 is a specific flow chart of step S320 in a preferred embodiment of a residual stress measurement method for ceramic materials in the present disclosure.

FIG. 6 is a specific flow chart of step S500 in a preferred embodiment of a residual stress measurement method for ceramic materials in the present disclosure.

### DETAILED DESCRIPTION OF THE EMBODIMENTS

In order to make the objectives, technical solutions and advantages of the present disclosure clearer, the present disclosure will be further described with reference to the attached figures and embodiments thereof. It shall be understood that, the embodiments described herein are only intended to illustrate but not to limit the present disclosure.

At present, there is no perfect technology to measure the residual stress of ceramic materials in the market. In the present disclosure, the residual stress of ceramic materials is successfully measured by introducing a laser engraving and using deformation released by the materials, so that the blank of residual stress measurement in the ceramic industry is filled.

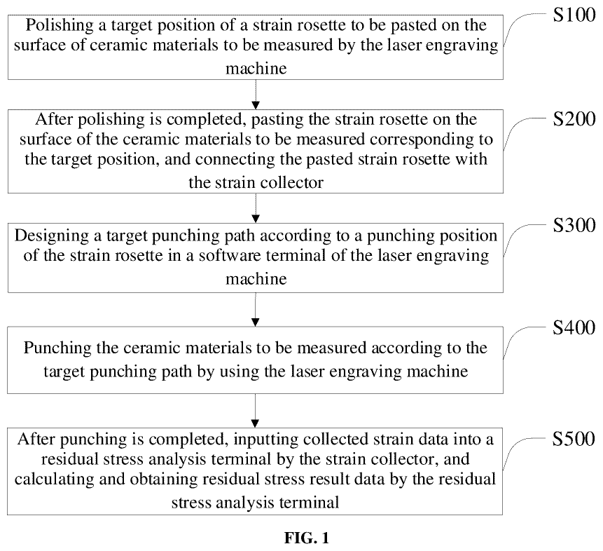

Referring to FIG. 1, FIG. 1 is a flow chart of a residual stress measurement method for ceramic materials in the present disclosure. A residual stress measurement method for ceramic materials is realized based on a residual stress measurement system for ceramic materials. The residual stress measurement system for ceramic materials includes a laser engraving machine, a strain collector, and a residual stress analysis terminal connected with the strain collector.

As shown in FIG. 1, a residual stress measurement method for ceramic materials in the embodiment of the present disclosure includes the following steps:
Step S100, polishing a target position of a strain rosette to be pasted on the surface of ceramic materials to be measured by the laser engraving machine.

In an implementation method, referring to FIG. 2, step S100 includes the following steps:
Step S110, placing an ice box on a workbench of a laser engraving machine, the ice box is filled with ice cubes;
Step S120, placing the ceramic materials to be measured on the ice box, and setting a position to be punched at a middle position of the ice box; and
Step S130, turning on the laser engraving machine, and polishing a target position of a strain rosette to be pasted on the surface of the ceramic materials to be measured.

The present disclosure can solve the application problem of residual stress measurement in the field of ceramic materials. The traditional blind hole method is difficult to punch holes on the surface of ceramic materials, and a thermal effect produced by punching also affects the testing accuracy and cannot meet the needs of residual stress testing of ceramic materials. Therefore, in the present disclosure, holes are punched by using a laser engraving machine, and an ice box is used for cooling, so that the influence of heat during punching on measurement results is avoided.

Specifically, an ice box filled with ice cubes is placed on the workbench of the laser engraving machine, ceramic materials are placed on the ice box, the required punching place is located in the middle position of the ice box, the laser engraving machine is turned on, and the position where the strain flower is required to stick on the surface of the ceramic materials is polished by the laser engraving machine. Wherein, during polishing, the laser wavelength is 355 nm, the pulse width is 5-30 ns, the frequency is 5-30 HZ, the power is 3-30 W, and the number of times of polishing is 1-5.

After step S100, step S200, after polishing is completed, pasting the strain rosette on the surface of the ceramic materials to be measured corresponding to the target position, and connecting the pasted strain rosette with the strain collector.

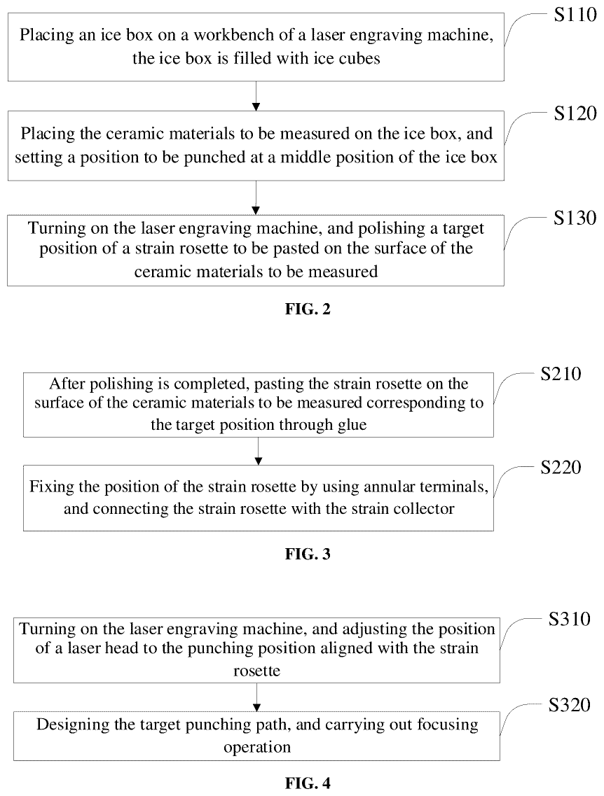

In an implementation method, referring to FIG. 3, step S200 specifically includes the following steps:
step S210, after polishing is completed, pasting the strain rosette on the surface of the ceramic materials to be measured corresponding to the target position through glue; and
step S220, fixing the position of the strain rosette by using annular terminals, and connecting the strain rosette with the strain collector.

Specifically, the strain rosette is pasted on the surface of the polished ceramic materials with glue, the position of the strain rosette is fixed by using the annular terminals, and the strain rosette is collected with the strain collector. The strain rosette includes three strain elements (sensitive grids), and the angles of the sensitive grids are 0°, 45° and 90°, respectively, and used for recording strain values in three directions.

After step S200, step S300, designing a target punching path according to a punching position of the strain rosette in a software terminal of the laser engraving machine.

Specifically, the residual stress analysis terminal is equipped with a residual stress analysis software, such as Sigma integrated testing software. The residual stress analysis terminal is connected with the strain collector before punching.

In an implementation method, before step S300, the method also includes the step of inputting the elastic modulus and Poisson's ratio of the ceramic materials to be measured in the residual stress analysis terminal on the residual stress analysis terminal. The elastic modulus and Poisson's ratio are actually measured values. For example, the elastic modulus and Poisson's ratio of building ceramic rock slabs are 70 GPa and 0.23, respectively. The elastic modulus and Poisson's ratio are used for calculating residual stress result data.

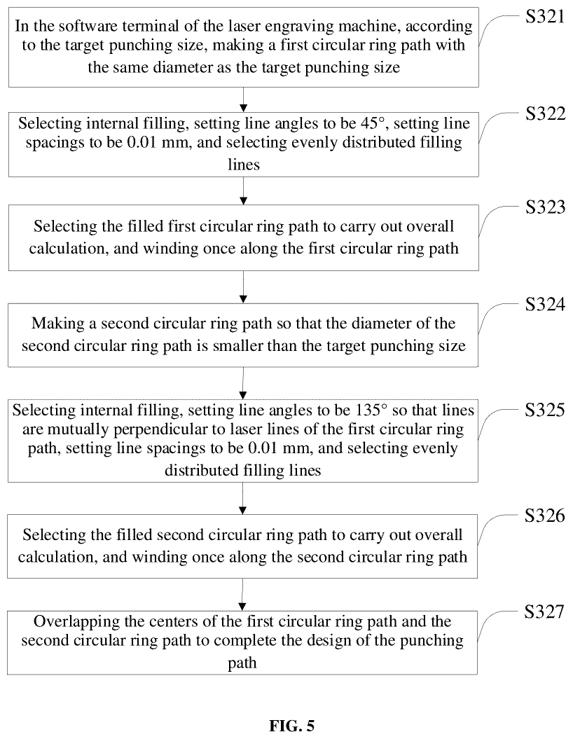

In an implementation method, referring to FIG. 4, step S300 specifically includes the following steps:
Step S310, turning on the laser engraving machine, and adjusting the position of a laser head to the punching position aligned with the strain rosette; and
Step S320, designing the target punching path, and carrying out focusing operation.

Specifically, the laser engraving machine is turned on, the position of a laser head is adjusted to the punching position aligned with the strain rosette, the punching path is designed, and focusing operation is carried out. The laser wavelength of the laser engraving machine is 355 nm, a 3D galvanometer scanning head is adopted, the diameter D of target punching size is 0.5-3 mm, and the depth of target punching size is 1.2×D.

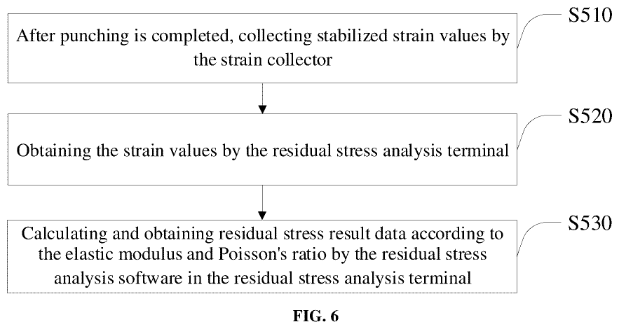

In an implementation method, referring to FIG. 5, the step of designing the target punching path in step S320 specifically includes the following steps:
Step S321, in the software terminal of the laser engraving machine, according to the target punching size, making a first circular ring path with the same diameter as the target punching size;
Step S322, selecting internal filling, setting line angles to be 45°, setting line spacings to be 0.01 mm, and selecting evenly distributed filling lines;
Step S323, selecting the filled first circular ring path to carry out overall calculation, and winding once along the first circular ring path;
Step S324, making a second circular ring path so that the diameter of the second circular ring path is smaller than the target punching size;
Step S325, selecting internal filling, setting line angles to be 135° so that lines are mutually perpendicular to laser lines of the first circular ring path, setting line spacings to be 0.01 mm, and selecting evenly distributed filling lines;
Step S326, selecting the filled second circular ring path to carry out overall calculation, and winding once along the second circular ring path; and
Step S327, overlapping the centers of the first circular ring path and the second circular ring path to complete the design of the punching path.

That is to say, according to required punching size (namely, target punching size), the first circular ring path with the same size as the target punching size is made. Internal filling is selected, the line angles are set to be 45°, the line spacings are set to be 0.01 mm, the evenly distributed filling lines are selected, the object (namely, the filled first circular ring path) is selected for overall calculation around the first circular ring path. Then, a second circular ring path of which the size is 0.05-0.2 mm smaller than the required punching size is made. Internal filling is selected, and the line angles are set to be 135°. Lines are mutually perpendicular to laser lines of the first circular ring path. The line spacings are set to be 0.01 mm, and the evenly distributed filling lines are selected. The object (namely, the filled second circular ring path is 0.05-0.2 mm smaller than the required punching size) is selected for overall calculation around the edge. The centers of the two circular ring paths are overlapped, and the two objects are placed at the same position, so that the design of the punching paths is completed. With the path designed by the method, the pore diameter with high verticality which can meet the test requirements can be manufactured.

After step S300, step S400, punching the ceramic materials to be measured according to the target punching path by using the laser engraving machine. Specifically, after focusing is completed, the laser engraving machine is turned on to punch holes.

In an implementation method, the residual stress measurement system for ceramic materials also includes an air draft device used for eliminating powder generated by the laser engraving machine during punching. Step S400 specifically includes the steps of punching the ceramic materials to be measured according to the target punching path by using the laser engraving machine, and turning on the air draft device at the same time. Wherein, the laser power of the laser engraving machine is 3-30 W, the diameter of focused pulsed beams is 0.02-0.05 mm, the pulse width is 5-30 ns, and the frequency is 5-30 HZ. The suction of the air draft device is 1-10 KPa, the air volume is 10-100 m³/h, and the distance between a suction opening of the air draft device and the laser drilling position is 0.1-1 m. The laser engraving machine is in a discontinuous working mode, and continuously works for 1-10 cycles each time according to set parameters. The depth of interaction is 0.005-0.05 mm each time, and the next operation is carried out at an interval of 10-60 seconds after each interaction until the depth of the target punching size is reached. That is to say, the working mode of the laser is performed step by step, and a laser is in a discontinuous processing mode according to the set parameters. The depth of interaction is 0.005-0.05 mm each time, and the next operation is carried out at an interval of 10-60 seconds after each interaction so as to reciprocate the operation until the depth of the hole is 1.2×D to complete the punching operation. The strain collector records the strain values in different directions after final stabilization.

After step S400, step S500, after punching is completed, inputting collected strain data into a residual stress analysis terminal by the strain collector, and calculating and obtaining residual stress result data by the residual stress analysis terminal.

In an implementation method, referring to FIG. 6, step S500 specifically includes the following steps:
Step S510, after punching is completed, collecting stabilized strain values by the strain collector;
Step S520, obtaining the strain values by the residual stress analysis terminal; and
Step S530, calculating and obtaining residual stress result data according to the elastic modulus and Poisson's ratio by the residual stress analysis software in the residual stress analysis terminal.

Specifically, after punching is completed, the strain values in three directions (0°, 45° and) 90° of the stabilized strain rosette are collected by the strain collector. Combined with the input elastic modulus and Poisson's ratio, the residual stress result data are automatically calculated by a calculation formula and Sigma comprehensive test software, so that the residual stress measurement at a punching position is completed.

In an implementation method, the residual stress result data include maximum principal stress, minimum principal stress, an included angle of 0 degree and equivalent stress. Through the residual stress result data, the representation problem of residual stress of ceramic materials is solved for guiding production, so that the deformation and cracking problems of ceramic materials in the later using process are solved.

In this way, a laser engraving machine is used for punching, and has the advantages of fast speed and accurate punching. The laser is a cold light source, and the punching heat influence is small. Combined with process settings, the thermal influence is reduced and the punching accuracy is improved, so that the accuracy of ceramic residual stress measurement is improved. By using the measurement method in the present disclosure, the residual stress of ceramic materials is measured to guide the production of the ceramic materials, the production quality of ceramic products can be improved, and risks and hidden dangers existing in the later using process are solved. According to the residual stress measurement method for ceramic materials formed by the present disclosure, the residual stress size of the ceramic materials can be accurately tested, and the blank of residual stress measurement for ceramic materials is filled around the world.

The present disclosure also provides a residual stress measurement system for ceramic materials, including:
a laser engraving machine, used for polishing a target position of a strain rosette to be pasted on the surface of the ceramic materials to be measured, and punching the ceramic materials to be measured according to the target punching path;
a strain collector, used for connecting a strain rosette pasted on the surface of the ceramic materials to be measured corresponding to the target position after polishing is completed, and inputting collected strain data into a residual stress analysis terminal after punching is completed;
a residual stress analysis terminal, connected with the strain collector and used for collecting released strain and calculating and obtaining residual stress result data; and
an air draft device, used for eliminating powder generated during punching of the laser engraving machine, specifically as mentioned previously.

In conclusion, the present disclosure provides a residual stress measurement method and system for ceramic materials. The residual stress measurement method is realized based on the residual stress measurement system for ceramic materials. The residual stress measurement method for ceramic materials includes a laser engraving machine, a strain collector, and a residual stress analysis terminal connected with the strain collector. The residual stress measurement method for ceramic materials includes the following steps: polishing a target position of a strain rosette to be pasted on the surface of the ceramic materials to be measured by using the laser engraving machine: after polishing is completed, pasting the strain rosette on the surface of the ceramic materials to be measured corresponding to the target position, and connecting the pasted strain rosette with the strain collector; designing a target punching path according to a punching position of the strain rosette in a software terminal of the laser engraving machine in a software terminal of the laser engraving machine; punching the ceramic materials to be measured according to the target punching path by using the laser engraving machine; after punching is completed. inputting collected strain data into the residual stress analysis terminal by the strain collector, and calculating and obtaining residual stress result data by the residual stress analysis terminal. The ceramic materials are punched by the laser engraving machine, and the residual stress result data are automatically calculated by the residual stress analysis terminal, so that the residual stress measurement of the ceramic materials is realized and can be used for the production guidance of the ceramic material, and then the production quality of ceramic products is improved to solve risks and hidden dangers existing in the later using process.

It should be understood that the application of the present disclosure is not limited to the examples described above, and these modifications or variations can be made according to the above description for those skilled in the art, all of which are intended to fall within the scope of the appended claims.

What is claimed is:

1. A residual stress measurement method for ceramic materials, the residual stress measurement method for ceramic materials being realized based on a residual stress measurement system for ceramic materials, wherein the residual stress measurement system for ceramic materials comprises a laser engraving machine, an ice box, a strain collector, and a residual stress analysis terminal connected with the strain collector;
the residual stress measurement method for ceramic materials comprises the following steps;
placing the ice box on a workbench of a laser engraving machine, the ice box is filled with ice cubes;
polishing a target position of a strain rosette to be pasted on the surface of ceramic materials to be measured by the laser engraving machine;
after polishing is completed, pasting the strain rosette on the surface of the ceramic materials to be measured corresponding to the target position, and connecting the pasted strain rosette with the strain collector;
designing a target punching path according to a punching position of the strain rosette in a software terminal of the laser engraving machine;
punching the ceramic materials to be measured according to the target punching path by using the laser engraving machine, the laser power is 3-30 W, the diameter of focused pulsed beams is 0.02-0.05 mm, the pulse width is 5-30 ns, the frequency is 5-30 HZ, a discontinuous processing mode is adopted, the depth of interaction is 0.005-0.05 mm every time, and the next operation is carried out at an interval of 10-60 seconds after each interaction, so that reciprocating interaction is carried out until the depth of the target punching size is reached, and punching operation is completed; and after punching is completed, inputting collected strain data into a residual stress analysis terminal by the strain collector, and calculating and obtaining residual stress result data by the residual stress analysis terminal.

2. The residual stress measurement method for ceramic materials according to claim 1, wherein the step of polishing a target position of a strain rosette to be pasted on the surface of the ceramic materials to be measured by the laser engraving machine comprises the following steps:
placing the ceramic materials to be measured on the ice box, and setting a position to be punched at a middle position of the ice box; and
turning on the laser engraving machine, and polishing a target position of a strain rosette to be pasted on the surface of the ceramic materials to be measured;
wherein during polishing, the laser wavelength is 355 nm, the pulse width is 5-30 ns, the frequency is 5-30 HZ, the power is 3-30 W, and the number of times of polishing is 1-5.

3. The residual stress measurement method for ceramic materials according to claim 1, wherein after polishing is completed, the step of pasting the strain rosette on the surface of the ceramic materials to be measured corresponding to the target position, and connecting the pasted strain rosette with the strain collector comprises the following steps:
after polishing is completed, pasting the strain rosette on the surface of the ceramic materials to be measured corresponding to the target position through glue; and fixing the position of the strain rosette by using annular terminals, and connecting the strain rosette with the strain collector;
wherein the strain rosette comprises three strain elements, and the angles of sensitive grids are 0°, 45° and 90°, respectively.

4. The residual stress measurement method for ceramic materials according to claim 3, wherein before the step of designing a target punching path according to a punching position of the strain rosette in a software terminal of the laser engraving machine, the method also comprises the following step:
inputting the elastic modulus and Poisson's ratio of the ceramic materials to be measured in the residual stress analysis terminal on the residual stress analysis terminal.

5. The residual stress measurement method for ceramic materials according to claim 1, wherein the step of designing a target punching path according to a punching position of the strain rosette in a software terminal of the laser engraving machine comprises the following steps:
turning on the laser engraving machine, and adjusting the position of a laser head to the punching position aligned with the strain rosette;
designing the target punching path, and carrying out focusing operation;
wherein the laser wavelength of the laser engraving machine is 355 nm, a 3D (three-dimensional) galvanometer scanning head is adopted, the diameter of target punching size is 0.5-3 mm, and the depth of target punching size is 1.2 times of the diameter.

6. The residual stress measurement method for ceramic materials according to claim 5, wherein the step of designing a target punching path specifically comprises the following steps:
in the software terminal of the laser engraving machine, according to the target punching size, making a first circular ring path with the same diameter as the target punching size;
selecting internal filling, setting line angles to be 45°, setting line spacings to be 0.01 mm, and selecting evenly distributed filling lines;
selecting the filled first circular ring path to carry out overall calculation, and winding once along the first circular ring path;
making a second circular ring path so that the diameter of the second circular ring path is smaller than the target punching size;
selecting internal filling, setting line angles to be 135° so that lines are mutually perpendicular to laser lines of the first circular ring path, setting line spacings to be 0.01 mm, and selecting evenly distributed filling lines;
selecting the filled second circular ring path to carry out overall calculation, and winding once along the second circular ring path; and
overlapping the centers of the first circular ring path and the second circular ring path to complete the design of the punching path.

7. The residual stress measurement method for ceramic materials according to claim 6, wherein the residual stress measurement system for ceramic materials also comprises an air draft device used for eliminating powder generated by the laser engraving machine during punching:
the step of punching the ceramic materials to be measured according to the target punching path by using the laser engraving machine comprises the following steps;
punching the ceramic materials to be measured according to the target punching path by using the laser engraving machine, and turning on the air draft device at the same time;
the suction of the air draft device is 1-10 KPa, the air volume is 10-100 m³/h, and the distance between a suction opening of the air draft device and the laser drilling position is 0.1-1 m.

8. The residual stress measurement method for ceramic materials according to claim 4, wherein after punching is completed, the step of inputting collected strain data into a residual stress analysis terminal by the strain collector, and calculating and obtaining residual stress result data by the residual stress analysis terminal comprises the following steps:
after punching is completed, collecting stabilized strain values by the strain collector;
obtaining the strain values by the residual stress analysis terminal; and
calculating and obtaining residual stress result data according to the elastic modulus and Poisson's ratio by the residual stress analysis software in the residual stress analysis terminal.

9. The residual stress measurement method for ceramic materials according to claim 1, wherein the residual stress result data comprise maximum principal stress, minimum principal stress, an included angle of 0 degree and equivalent stress.

10. A residual stress measurement system for ceramic materials, comprising:
a laser engraving machine, used for polishing a target position of a strain rosette to be pasted on the surface of ceramic materials to be measured and punching the ceramic materials to be measured according to the target punching path, the laser power is 3-30 W, the diameter of focused pulsed beams is 0.02-0.05 mm, the pulse width is 5-30 ns, the frequency is 5-30 HZ, a discontinuous processing mode is adopted, the depth of interaction is 0.005-0.05 mm every time, and the next operation is carried out at an interval of 10-60 seconds after each interaction, so that reciprocating interaction is carried out until the depth of the target punching size is reached, and punching operation is completed; and
an ice box, capable of accommodating ice cubes, and used for cooling down during punching of the laser engraving machine to reduce a thermal effect produced by punching and improve the accuracy of strain data;
a strain collector, used for connecting a strain rosette pasted on the surface of the ceramic materials to be measured corresponding to the target position after polishing is completed, and inputting collected strain data into a residual stress analysis terminal after punching is completed;
a residual stress analysis terminal, connected with the strain collector and used for collecting released strain and calculating and obtaining residual stress result data; and
an air draft device, used for eliminating powder generated during punching of the laser engraving machine.
